(19) United States
(12) Patent Application Publication
(10) Pub. No.: US 2025/0378573 A1
(43) Pub. Date: Dec. 11, 2025

(54) FIRE POSITIONING SYSTEM AND FIRE POSITIONING METHOD

(71) Applicant: RozeAI Korea Co., Ltd., Seongnam-si (KR)

(72) Inventors: Young-Jin CHO, Seongnam-si (KR); Chang-Sik JUNG, Seoul (KR)

(21) Appl. No.: 19/234,539

(22) Filed: Jun. 11, 2025

(30) Foreign Application Priority Data
Jun. 11, 2024 (KR) 10-2024-0075390

(51) Int. Cl. G06T 7/70; G06T 7/50; G06V 20/52; G08B 17/12

(52) U.S. Cl. CPC G06T 7/70; G06T 7/50; G06V 20/52; G08B 17/125; G06T 2207/10048; G06T 2207/20084; G06T 2207/30232

(57) ABSTRACT

A fire positioning system is disclosed. The fire positioning system comprises a sensing unit configured to measure temperature and smoke information to generate fire data, a first imaging unit configured to capture an image to generate a first image, a second imaging unit configured to capture an infrared image to generate a second image, and a gateway configured to receive the fire data, the first image, and the second image.

### CROSS-REFERENCE TO RELATED APPLICATIONS

This application claims priority under 35 U.S.C. § 119 to Korean Patent Application No. 10-2024-0075390 filed on Jun. 11, 2024 in the Korean Intellectual Property Office, the disclosure of which is incorporated by reference herein in its entirety.

### BACKGROUND

The present disclosure relates to a fire positioning system and a fire positioning method with improved reliability.

In general, a fire causes significant damage not only to human lives but also economically. Once the fire occurs, it is a tremendous disaster that requires considerable time and cost to recover from.

Therefore, it is crucial to quickly identify situations where the fire has occurred or where a fire situation is suspected and respond accordingly.

Typically, flame detectors are used to detect fires, but due to the cost of flame detectors and the need for early detection of the fires, thermal cameras have recently been widely used. High-performance thermal cameras use technology that simultaneously measures a distance between the camera and an object to accurately measure the object's temperature, allowing an system to determine a distance from the camera's origin to the detected fire.

### SUMMARY

The present disclosure aims to provide a fire positioning system and a fire positioning method with enhanced reliability.

A fire positioning system comprises a sensing unit configured to measure temperature and smoke information to generate fire data, a first imaging unit configured to capture an image to generate a first image, a second imaging unit configured to capture an infrared image to generate a second image, and a gateway configured to receive the fire data, the first image, and the second image. The gateway comprises, a space coordinate definition unit configured to define a space where the sensing unit, the first imaging unit, and the second imaging unit are located in three-dimensional coordinates, an image analysis unit configured to detect a fire based on the first image to generate first image data, a fire determination unit configured to determine an occurrence of the fire based on the first image data and the second image to generate a fire event signal, and an origin positioning unit configured to receive the first image data and the fire event signal. The origin positioning unit generates fire coordinates in accordance with the three-dimensional coordinates by using both a depth estimation algorithm and a back projection algorithm.

For example, the depth estimation algorithm estimates a distance between the first imaging unit and a target by using a convolutional neural network.

For example, the depth estimation algorithm estimates the distance by using the first image data for the first images.

For example, the back projection algorithm generates the fire coordinates based on an optical center of the first imaging unit, the distance, and the three-dimensional coordinates.

For example, the fire positioning system further comprises a server configured to communicate with the gateway, the gateway further comprises a communication unit and a power supply, and the communication unit transmits the fire event signal and the fire coordinates to the server.

For example, the power supply is configured to supply power to the space coordinate definition unit, the fire determination unit, and the origin positioning unit.

For example, the server calculates a fire index, which is an index of probability of a fire at a location corresponding to the fire coordinates based on the fire data, the first image, and the second image.

For example, the fire determination unit determines a false fire alarm by comparing the first image data of an n-th frame with the first image data of an n+1-th frame.

For example, the gateway receives the first image and the second image on a frame-by-frame basis, and the origin positioning unit calculates the fire coordinates for each frame.

For example, the gateway further comprises a memory, and the memory stores location coordinates defined according to the three-dimensional coordinates for each of the sensing unit, the first imaging unit, and the second imaging unit.

A fire positioning method comprises, defining a space, in which a first imaging unit configured to capture an image to generate a first image and a second imaging unit configured to capture an infrared image to generate a second image are located, in three-dimensional coordinates, detecting a fire based on the first image to generate first image data, determining an occurrence of the fire based on the first image data and the second image to generate a fire event signal, and receiving the first image data and the fire event signal, and generating fire coordinates based on the three-dimensional coordinates by using both a depth estimation algorithm and a back projection algorithm.

For example, the depth estimation algorithm estimates a distance between the first imaging unit and a target by using a convolutional neural network.

For example, the depth estimation algorithm estimates the distance by using the first image data for the first images.

For example, the back projection algorithm generates the fire coordinates based on an optical center of the first imaging unit, the distance, and the three-dimensional coordinates.

For example, generating the fire event signal further comprises determining a false fire alarm by comparing the first image data of an n-th frame with the first image data of an n+1-th frame.

### BRIEF DESCRIPTION OF THE FIGURES

The above and other objects and features of the present disclosure will become apparent by describing in detail embodiments thereof with reference to the accompanying drawings.

FIG. 1 illustrates a fire positioning system according to an embodiment of the present disclosure.

FIG. 2 is a block diagram of a gateway according to an embodiment of the present disclosure.

FIG. 3 is a flowchart illustrating a fire positioning method according to an embodiment of the present disclosure.

FIG. 4 illustrates a depth estimation algorithm according to an embodiment of the present disclosure.

FIG. 5 illustrates a back projection algorithm according to an embodiment of the present disclosure.

FIG. 6a and FIG. 6b illustrate first image data of each of the plurality of frames according to an embodiment of the present disclosure.

### DETAILED DESCRIPTION

In this document, when any component (or region, layer, part, etc.) is mentioned as being ‘on,’ ‘connected to,’ or ‘coupled with’ another component, it means that the component may be directly placed/connected/coupled on the other component, or that a third component may be placed between them.

The same reference numerals refer to the same components. Additionally, in the drawings, the thickness, proportions, and dimensions of the components are exaggerated for effective description of the technical content. The term ‘and/or’ includes all combinations that may be defined by the associated components.

The terms ‘first,’ ‘second,’ etc., may be used to describe various components, but these components should not be limited by these terms. These terms are used only to distinguish one component from another. For example, without departing from the scope of the present disclosure, a first component may be named as the second component, and similarly, the second component may be named as the first component. Singular expressions include plural expressions unless the context clearly indicates otherwise.

Furthermore, the terms ‘below,’ ‘lower,’ ‘above,’ ‘upper,’ etc., are used to describe the relationships between components shown in the drawings. These terms are relative concepts and are described based on the direction shown in the drawings.

The terms ‘comprise’ or ‘include’ are meant to indicate that the features, numbers, steps, actions, components, parts, or combinations of them described in this document exist and should not be interpreted as excluding the presence or addition of other features, numbers, steps, actions, components, parts, or combinations of them.

Unless otherwise defined, all terms used in this document (including technical and scientific terms) have the same meaning as generally understood by those skilled in the art to which the present disclosure pertains. Additionally, terms defined in commonly used dictionaries should be interpreted in a way that aligns with their meaning in the context of the relevant technology, and unless explicitly defined here, they should not be interpreted in an overly idealistic or excessively formal manner.

Hereinafter, embodiments of the present disclosure will be described with reference to the drawings.

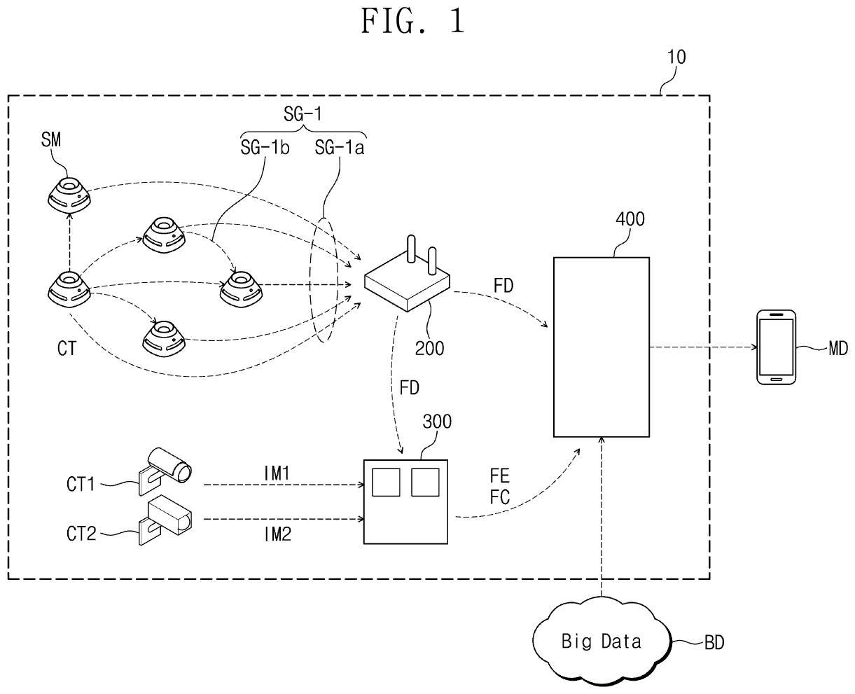

FIG. 1 illustrates a fire positioning system according to an embodiment of the present disclosure.

Referring to FIG. 1, the fire positioning system 10 may include a plurality of sensing units SM, a relay unit 200, a first imaging unit CT1, a second imaging unit CT2, a gateway 300, and a server 400.

Each of the plurality of sensing units SM may detect information on heat, smoke, temperature, humidity, and gas. The gas may include, for example, carbon monoxide. Each of the plurality of sensing units SM may be referred to as a multi-sensor.

Each of the plurality of sensing units SM may generate fire data FD. The fire data FD may include information on at least one of current temperature, smoke, humidity, flame, or gas at a location where the plurality of sensing units SM are installed.

Additionally, each of the plurality of sensing units SM may detect whether the fire has occurred.

Each of the plurality of sensing units SM may generate a fire detection signal SG-1. The fire detection signal SG-1 may include the fire data FD.

The plurality of sensing units SM may transmit the fire detection signal SG-1 to adjacent sensing units SM and/or the relay unit 200.

The fire detection signal SG-1 may include a first signal SG-1a and a second signal SG-1b. The first signal SG-1a may be a signal generated by the sensing unit SM that detects an occurrence of the fire. The second signal SG-1b may be a signal amplified by the sensing unit SM.

The method for transmitting and receiving the fire detection signal SG-1 and the fire data FD may utilize Radio Frequency (RF) communication. The RF communication method may be a communication method that exchanges information by radiating radio frequencies. As a broadband communication method using frequencies can have high stability with less impact from climate and environmental factors. The RF communication method may also support voice or other additional features and may offer fast transmission speeds. For example, the RF communication method may use frequencies in a range of 447 MHz to 924 MHz. However, this is merely an example, and in one embodiment of the present disclosure, communication methods such as Ethernet, WiFi, LoRa, M2M, 3G, 4G, LTE, LTE-M, Bluetooth, or WiFi Direct may also be used.

In one embodiment of the present disclosure, the RF communication method may include LBT (Listen Before Transmission) communication method. This is a frequency selection method that determines whether a selected frequency is being used by another system, and selects a different frequency if it is occupied. For example, a node, being intended to transmit, first listens to a medium to determine whether it is idle, and then, before transmission, may send a backoff protocol. By using the LBT communication method to distribute data processing, signal collisions within the same frequency band can be prevented.

The relay unit 200 may communicate with the plurality of sensing units SM via RF communication. The relay unit 200 may receive the fire detection signal SG-1 including the fire data FD.

The first imaging unit CT1 may capture an image to generate a first image IM1. The first imaging unit CT1 may generate the first image IM1 in real-time. The first imaging unit CT1 may include a CCTV.

The RF communication method may be used to transmit the first image IM1.

The second imaging unit CT2 may capture an infrared image to generate the second image IM2. The second imaging unit CT2 may generate the second image IM2 in real-time. The second imaging unit CT2 may include a thermal camera.

The RF communication method may be used to transmit the second image IM2.

The gateway 300 may receive the fire data FD, the first image IM1, and the second image IM2.

The gateway 300 may generate a fire event signal FE and fire coordinates FC. The fire event signal FE may include information indicating that the fire has occurred.

The server 400 may communicate with the gateway 300 and the relay unit 200. The server 400 may receive the fire event signal FE and the fire coordinates FC from the gateway 300. The server 400 may receive the fire data FD from the relay unit 200.

The server 400 may combine the fire event signal FE and the fire data FD to determine the occurrence of the fire. That is, the server 400 may determine the fire by using a multimodal approach.

Unlike the present disclosure, in the case of using a single model, with an image-based model, there may be misinterpretation of non-fire events, such as a camera flash being mistaken for the fire, or a reduction in reliability when the fire's flames are large. In the case of a temperature-based model, it may be suitable for fixed environments, but may not be suitable for logistics warehouses where objects frequently move. With a smoke-based model, there may be misinterpretation of the fire if the air contains a high level of dust or humidity. However, according to the present disclosure, the server 400 may receive data from the sensing units SM, which sense heat, smoke, temperature, humidity, and gases, from the first imaging unit CT1, which captures images, and from the second imaging unit CT2, which captures infrared images. The server 400 may combine these data to determine the occurrence of the fire. As a result, false fire alarms can be easily identified, and the reliability of fire detection can be improved.

The server 400 may receive big data BD from an external big data server. The big data BD may be periodically updated. The big data BD refers to data that exceeds an ability of general software tools to collect, manage, and process within a permissible hardening time, and can be used as a mean to predict a diversified society. This large volume of data can provide more insights than the limited data available traditionally.

The big data BD may include information on buildings, places, and facilities. For example, the big data BD may include information on movie theaters, traditional market buildings, museums, army headquarters, air force headquarters, warehouses, shooting ranges, military barracks, boilers, turbines, or thermal power plants.

The big data BD may also include surrounding environmental data. For example, the surrounding environmental data may include at least one of data corresponding to fire occurrence probabilities by date, data corresponding to fire occurrence probabilities by time, data corresponding to fire occurrence probabilities by location, data corresponding to fire occurrence probabilities by temperature, data corresponding to fire occurrence probabilities by humidity, data corresponding to fire occurrence probabilities by weather, data corresponding to fire occurrence probabilities by industry, or data corresponding to fire occurrence probabilities by user.

For example, the data corresponding to fire occurrence probabilities by date may include fire occurrence probabilities by day of the week and/or fire occurrence probabilities by month. The data corresponding to fire occurrence probabilities by time may include fire occurrence probabilities separated into early morning, morning, afternoon, evening, or late night, etc. The data corresponding to fire occurrence probabilities by location may include fire occurrence probabilities categorized by urban, mountain, beach, or rural areas, etc. The data corresponding to fire occurrence probabilities by temperature may include fire occurrence probabilities categorized by spring, summer, fall, or winter. The data corresponding to fire occurrence probabilities by humidity may include fire indices for specific humidity levels. The data corresponding to fire occurrence probabilities by weather may include fire occurrence probabilities for clear, cloudy, or rainy days, etc. The data corresponding to fire occurrence probabilities by industry may include fire occurrence probabilities categorized by home, restaurant, factory, or office, etc. The data corresponding to fire occurrence probabilities by user may include fire occurrence probabilities categorized by age, occupation, or gender, etc.

The server 400 may calculate a fire index, which is an index representing the probability of the fire at the location corresponding to the fire coordinates FC, based on the fire data FD, the first image IM1, the second image IM2, and big data BD.

The fire index may be set differently depending on a unit, such as a unit area, a work space in a warehouse, or a logistics center. The unit area may be defined as a single rack in the warehouse, and the work space may consist of a plurality of racks. In other words, the fire index can be built for each space or facility. If the fire index exceeds a predetermined value, the server 400 may determine that the fire has occurred in the area of the fire coordinates FC, considering the fire event signal FE and the fire data FD as appropriate signals.

The server 400 may determine the probability of fire detection at a specific location based on the fire index and the fire coordinates FC. This allows the server 400 to easily determine false fire alarms for specific locations.

If the server 400 determines that the fire has occurred, it may send an alarm to the user's device MD. For example, the device MD may be deployed at a fire station, stakeholders at the location of the fire, a disaster control center (or public safety-related institutions), or a building's control room. The user may receive fire warning messages or alarms in a form of text messages, video messages, or voice messages through the device MD.

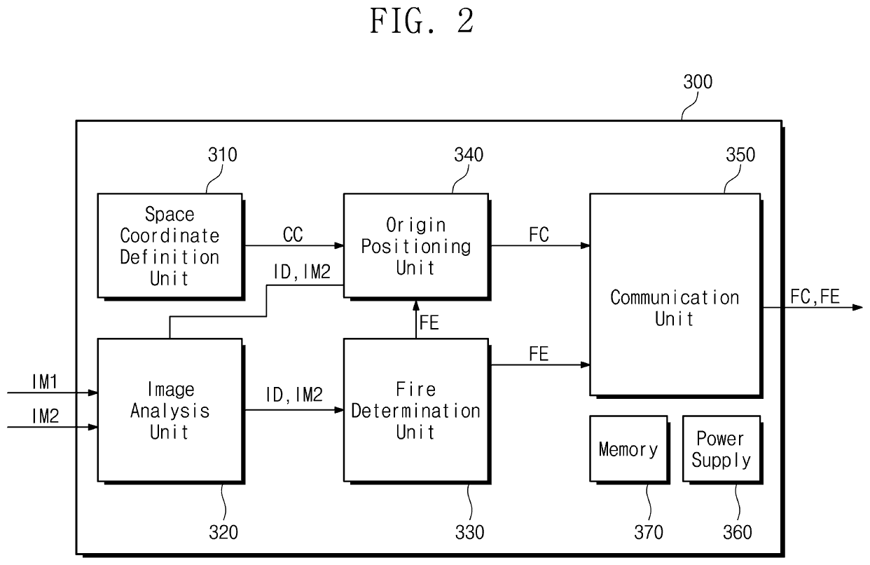

FIG. 2 is a block diagram of a gateway according to an embodiment of the present disclosure.

Referring to FIG. 1 and FIG. 2, the gateway 300 may include a space coordinate definition unit 310, an image analysis unit 320, a fire determination unit 330, an origin positioning unit 340, a communication unit 350, a power supply unit 360, and a memory 370.

The space coordinate definition unit 310 may define the space where the sensing units SM, the first imaging unit CT1, and the second imaging unit CT2 are located in three-dimensional coordinates. For example, the space may be defined as a logistics warehouse.

In the case of a logistics warehouse, due to high storage density and vertical storage methods, there is a high possibility that a large fire could occur. As a result, early response to fires in logistics warehouses is essential.

Additionally, in the case of a logistics warehouse, the structure is simple, making it easy to represent the space with coordinates. For example, the logistics warehouse may have a cubic structure, and the space coordinate definition unit 310 may express the width, length, and height of the logistics warehouse in three-dimensional coordinates. The three-dimensional coordinates may be expressed using the x, y, and z axes.

The space coordinate definition unit 310 may generate a location coordinate CC for each of the sensing unit SM, the first imaging unit CT1, and the second imaging unit CT2 based on the three-dimensional coordinates. The space coordinate definition unit 310 may transmit the location coordinate CC to the origin positioning unit 340.

The image analysis unit 320 may receive the first image IM1 and the second image IM2 on a frame-by-frame basis. The image analysis unit 320 may generate first image data ID based on the first image IM1.

The image analysis unit 320 may include a learning model. The learning model may be an artificial intelligence that uses machine learning to determine whether the fire exists based on the first image IM1. The artificial intelligence may refer to artificial intelligence itself or the methodologies used to create it, and machine learning may refer to the methodologies for defining and solving various problems in the field of artificial intelligence. The machine learning algorithm can be defined as an algorithm that improves the performance of a task through continuous experience with that task.

The learning model may include a deep neural network. The deep neural network may be designed to simulate the structure of the human brain within the learning model. The deep neural network, as one of the models used in machine learning, may be composed of artificial neurons (nodes) that form a network through synaptic connections, and it may represent the overall model that has the ability to solve problems. The deep neural network may be defined by the connection patterns between neurons in different layers, the learning process that updates model parameters, and the activation functions that generate output values.

The deep neural network may include an input layer, an output layer, and at least one hidden layer. Each layer may include one or more neurons, and the deep neural network may include synapses connecting the neurons. Each neuron in the deep neural network may output the activation function value for signals, weights, and biases input through synapses.

The deep neural network may be trained according to supervised learning. The goal of the supervised learning may be to find a predetermined answer through the algorithm. Therefore, the deep neural network based on the supervised learning may include a form that infers a function from the training data. In the supervised learning, labeled samples may be used during training. The labeled sample may refer to the target output value that the deep neural network must infer when the training data is input into the deep neural network.

The algorithm may receive a series of training data and the corresponding target output values, may compare the actual output values for the input data with the target output values, and may identify errors through learning. Based on the results, the algorithm can be modified.

That is, the image analysis unit 320 may detect whether the image captured in the first image IM1 contains the fire by using artificial intelligence. For example, the image analysis unit 320 may classify and highlight the fire in the first image IM1 by using the learning model. The image analysis unit 320 may generate the first image data ID with the classified fire.

The image analysis unit 320 may transmit the first image data ID to the fire determination unit 330 and the origin positioning unit 340.

The fire determination unit 330 may determine the presence of the fire based on the first image data ID and the second image IM2 and generate the fire event signal FE. The fire determination unit 330 may combine the first image data ID and the second image IM2 to determine whether the fire is present in the image. That is, the gateway 300 may utilize the first imaging unit CT1 and the second imaging unit CT2 in a multimodal manner.

If the fire determination unit 330 determines that there is the fire in the image, the fire determination unit 330 may generate the fire event signal FE and transmit the fire event signal FE to the origin positioning unit 340 and the communication unit 350.

The origin positioning unit 340 may receive the location coordinate CC, the first image data ID, the second image IM2, and the fire event signal FE.

The origin positioning unit 340 may detect whether the fire has occurred based on the fire event signal FE. The origin positioning unit 340 may use both a depth estimation algorithm and a back projection algorithm to generate the fire coordinates FC according to the three-dimensional coordinates. That is, the origin positioning unit 340 may calculate the location of the fire as the fire coordinates FC. The origin positioning unit 340 may calculate the fire coordinates FC for each frame.

The origin positioning unit 340 may transmit the fire coordinates FC to the communication unit 350.

The communication unit 350 may receive the fire event signal FE and the fire coordinates FC. The communication unit 350 may communicate with the server 400. The communication unit 350 may include a wireless communication unit and a wired communication unit.

The wireless communication unit may communicate with the server 400 using RF communication.

The wired communication unit may communicate with the server 400 using Ethernet.

The power supply unit 360 may supply power to the space coordinate definition unit 310, the image analysis unit 320, the fire determination unit 330, the origin positioning unit 340, and the communication unit 350. The power supply unit 360 may receive power from an external source. The power supply unit 360 may further include a battery to prepare for power supply issues caused by the fire. The battery may be used as a backup power source.

The memory 370 may store the location coordinates CC. Additionally, the memory 370 may store the algorithms.

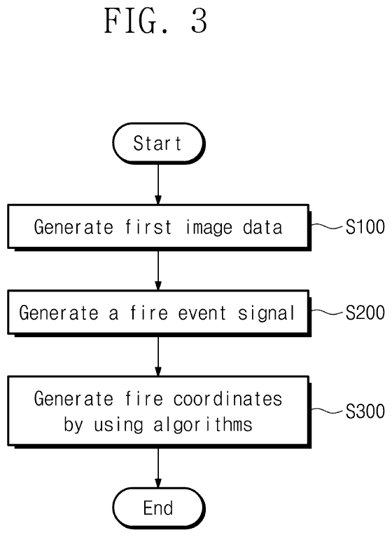

FIG. 3 is a flowchart illustrating a fire positioning method according to an embodiment of the present disclosure.

Referring to FIG. 1 through FIG. 3, the fire positioning method may comprise defining the space where the first imaging unit CT1 which captures images to generate the first image IM1, and the second imaging unit CT2 which captures infrared images to generate the second image IM2, are located in three-dimensional coordinates; detecting the fire based on the first image IM1 and generating first image data ID (S100); determining the occurrence of the fire based on the first image data ID and the second image IM2 and generating the fire event signal FE (S200); and generating the fire coordinates FC by using both the depth estimation algorithm and the back projection algorithm (S300).

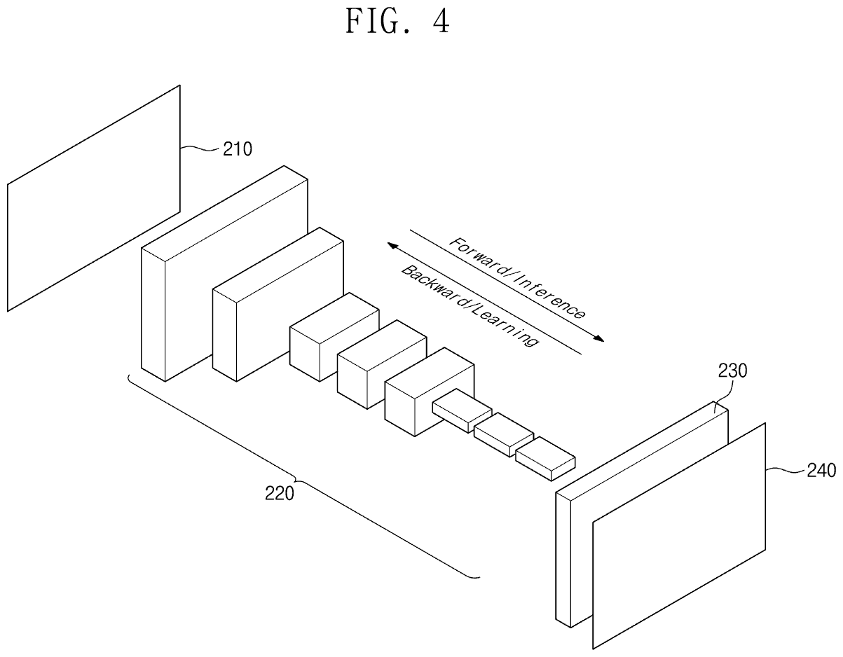

FIG. 4 illustrates the depth estimation algorithm according to an embodiment of the present disclosure.

Referring to FIG. 1, FIG. 2, and FIG. 4, the origin positioning unit 340 may estimate a distance between the first imaging unit CT1 and a target by using the depth estimation algorithm, which employs a deep neural network. In this case, the target may be the fire.

The deep neural network may be designed to simulate the structure of the human brain within the origin positioning unit 340. The deep neural network, as a model used in machine learning, may be composed of artificial neurons (nodes) that form a network through synaptic connections and may represent a model with problem-solving capabilities. The deep neural network may be defined by connection patterns between neurons in different layers, a learning process that updates model parameters, and activation functions that generate output values.

The deep neural network may include an input layer, an output layer, and at least one hidden layer. Each layer may contain one or more neurons, and the deep neural network may include synapses that connect neurons. Each neuron in the deep neural network may output the activation function value for the signals, weights, and biases input through synapses.

The deep neural network may be trained according to the supervised learning. The goal of the supervised learning may be to find the predetermined answer through the algorithm. Therefore, the deep neural network based on the supervised learning may include a form that infers a function from training data. In the supervised learning, labeled samples may be used during training. The labeled sample may refer to the target output value that the deep neural network must infer when the training data is input into the deep neural network.

The algorithm may receive a series of training data and the corresponding target output values, and by comparing the actual output values for the input data with the target output values through learning, errors may be identified, and based on the results, the algorithm can be modified.

The output of the supervised learning may include semantic segmentation. The semantic segmentation may refer to a technique that performs pixel-level estimation to separate objects into meaningful units. The semantic segmentation may involve distinguishing each object that constitutes the first image data ID input into the algorithm at the pixel level. For example, the distance between the first imaging unit CT1 and the target may be estimated from the labeled data 240.

The deep neural network may include a Fully Convolutional Network (FCN), Convolutional Neural Network (CNN), Recurrent Neural Network (RNN), Deep Belief Network (DBN), or Restricted Boltzmann Machine (RBM). However, this is exemplary, and the deep neural network according to one embodiment of the present disclosure is not limited to these. Below, the deep neural network is described as the Convolutional Neural Network.

The input image 210, the Convolutional Neural Network 220, the activation map 230 output from the Convolutional Neural Network 220, and the labeled data 240 are illustrated.

The input image 210 may include the first image data ID. The input image 210 may be provided as a single image. The input image 210 may include a depth map for the single image.

The convolution layers in the Convolutional Neural Network 220 may be used to extract the distance to the target from the single input image 210. Each convolution layer may receive data, process the input data, and generate output data from the layer. The data output from the convolution layer may be data generated by combining the input data with one or more filters.

The Convolutional Neural Network 220 may be an architecture designed for depth estimation. The architecture may include convolution layers to extract features from the first image data ID, pooling layers to reduce the feature map, and an output layer to estimate depth based on these.

The initial convolution layers may operate to extract low-level simple features from the input. The following convolution layers may operate to extract more complex high-level features compared to the initial convolution layers.

The data output from each convolution layer may be referred to as the feature map.

As the input image 210 passes through multiple layers within the Convolutional Neural Network 220, the size of the activation map may decrease. Since semantic segmentation involves pixel-level estimation of objects, to perform pixel-level estimation, the results of the reduced-size activation map must be resized back to the size of the input image 210. Methods for resizing the activation map back to the input image 210's size may include techniques like bilinear interpolation, deconvolution, or skip layer techniques. The final output activation map 230 in the Convolutional Neural Network 220 may have the same size as the input image 210. Therefore, the activation map 230 may preserve the positional information of the objects. The process of the Convolutional Neural Network 220 receiving the input image 210 and outputting the activation map 230 is called forward inference.

The designed Convolutional Neural Network 220 may perform a learning process. In this case, the network may be optimized using the first input data ID and the actual depth map. The learning process may involve adjusting the weights of the network to minimize the loss function.

For example, the learning method through backpropagation may start from the input layer, obtain the output value through the output layer, compare it with the target label value, and if there is an error, propagate the values backward from the output layer to the input layer, updating the weights of the nodes in the learning network according to the calculated loss. The training dataset provided to the Convolutional Neural Network 220 may be defined as ground truth data or labeled data 240. According to one embodiment of the present disclosure, the training dataset may consist of thousands to tens of thousands of still images. The labeled data 240 may include the actual depth map.

Once the Convolutional Neural Network 220 has performed the learning process using the input image 210, a trained model with optimized parameters may be created. When unlabeled data is input into the trained model, it may predict the corresponding labeled data for the inputted data.

The Convolutional Neural Network 220 may output the activation map 230. Based on the activation map 230, the distance between the first imaging unit CT1 and the target can be estimated.

The depth estimation algorithm using the Convolutional Neural Network 220 may use deep learning techniques to learn the features of complex input images 210 and, based on that learning, estimate the depth of the objects.

According to the present disclosure, the location of the fire's origin can be measured using the Convolutional Neural Network 220. When using the Convolutional Neural Network 220, the depth can be estimated using the first image data ID for a first image IM1 from a single first imaging unit CT1. This enables the installation of the fire positioning system 10 at a minimal cost. Moreover, the Convolutional Neural Network 220 may receive the first image data ID in real time, allowing for rapid depth estimation. In fire situations that require swift response, the distance between the fire and the first imaging unit CT1 can be estimated, the fire coordinates FC can be quickly calculated based on the location coordinates CC and the distance by using back-projection algorithm, which will be described later. That is, the origin positioning unit 340 may calculate and output the fire coordinates FC of the location where the fire has occurred. Therefore, the fire positioning system 10 with improved speed and reliability can be provided.

The second image IM2 received from the second imaging unit CT2 may include the distance between the second imaging unit CT2 and the object. For example, the second imaging unit CT2 may include a high-performance thermal camera. The high-performance thermal camera may include technology that utilizes the distance between the camera and the object to measure the temperature of the object accurately. The second imaging unit CT2 may use this technology to obtain the distance from the second imaging unit CT2 to the detected fire.

The origin positioning unit 340 may accurately estimate the distance to the fire from the imaging units CT1, CT2 by combining the first distance from the depth estimation algorithm of the first image data ID and the second distance detected from the second image IM2.

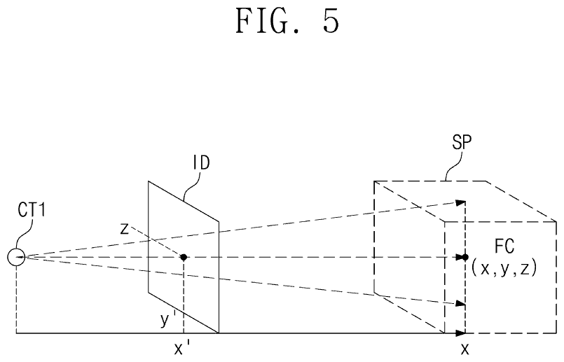

FIG. 5 illustrates a back projection algorithm according to an embodiment of the present disclosure.

Referring to FIGS. 1, 2, and 5, the origin positioning unit 340 may generate the fire coordinates FC based on the optical center x′, the distance x between the first imaging unit CT1 and the object, and the three-dimensional coordinates using the back-projection algorithm.

By using the measured distance x from the fire to the optical center x′ of the first imaging unit CT1, and the location coordinates CC of the first imaging unit CT1, the fire coordinates FC (x, y, z) can be calculated through trigonometric functions. The actual space SP where the fire has occurred can be defined as three-dimensional coordinates by the space coordinate definition unit 310.

The origin positioning unit 340 may receive the first image data ID converted from the first image IM1 by the image analysis unit 320.

The back-projection algorithm allows for learning a model of an object or pattern related to the fire. By using the trained model, back-projection can be performed to find parts of the first image data ID that resemble the model. The back-projection calculates the similarity between the model and each pixel of the first image data ID, generating a back-projection image. The back-projection image can be used to highlight areas that match the model.

Threshold processing may be performed to identify areas with high similarity to the model in the back-projection image. In this case, the coordinates of the pixels that match the model in the threshold-processed back-projection image can be extracted. The model may be the fire, and the coordinates of the pixels that match the model may be the fire image coordinates (y′, z′).

The fire captured in the first image data ID may have the fire image coordinates (y′, z′).

The distance x between the first imaging unit CT1 and the object may be calculated using the previously described depth estimation algorithm and the second image IM2.

By utilizing the proportional relationship between the optical center x′ of the first imaging unit CT1 and the distance x between the first imaging unit CT1 and the object, the fire coordinates FC (x, y, z) may be calculated from the fire image coordinates (y′, z′).

According to the present disclosure, the depth estimation algorithm may receive the first image data ID in real time to quickly estimate the distance x between the fire and the first imaging unit CT1. The back-projection algorithm, being based on simple operations, allows real-time tracking and recognition of the fire and quick calculation of the fire coordinates FC. Through this, the origin positioning unit 340 may calculate and output the fire coordinates FC representing the location of the fire. The gateway 300 may specify and output the fire coordinates FC of the fire's location. The user or server 400 may clearly identify the location of the fire and take subsequent actions. Therefore, the fire positioning system 10 with improved reliability can be provided.

Furthermore, according to the present disclosure, based on the fire coordinates FC, the location of the fire can be clearly identified. The server 400 may calculate the probability of the fire occurring at this location using the fire index. This enhances the firefighting capability in the early stages of the fire and can prevent the fire from spreading. Therefore, the fire positioning system 10 with improved reliability can be provided.

The server 400 stores the fire location and the type of fire occurring at that location as data based on the fire coordinates FC, and by monitoring and analyzing this data, the causes and patterns of the fire can be accurately understood. This information can later be used for effective prevention methods and improvements to the system.

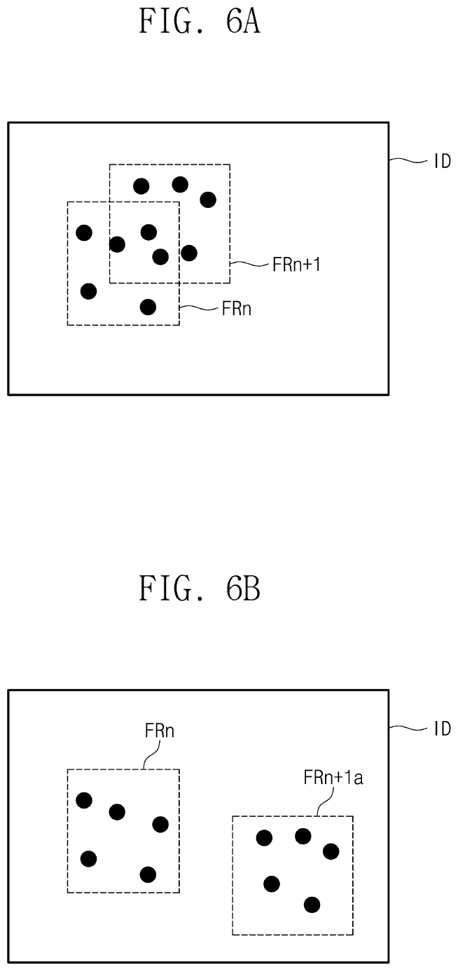

FIG. 6a and FIG. 6b illustrate first image data of each of the plurality of frames according to an embodiment of the present disclosure.

Referring to FIG. 1, FIG. 2, FIG. 6a, and FIG. 6b, the fire determination unit 330 may determine a false alarm by comparing the first image data ID from the n-th frame FRn with the first image data ID from the n+1-th frame FRn+1, FRn+1a.

In the first image data ID, the dots represent the fire classified in the first image IM1, shown as an example using a learned model. The fire determination unit 330 may use multiple frames of the first image data ID in a time-series to determine the fire.

Referring to FIG. 6a, at least some of the regions defined by the dots from the first image data ID measured in the n-th frame FRn and the first image data ID measured in the n+1-th frame FRn+1 may overlap. In this case, the fire determination unit 330 may determine the occurrence of the fire and generate the fire event signal FE.

Referring to FIG. 6b, the regions defined by the dots from the first image data ID measured in the n-th frame FRn and the first image data ID measured in the n+1-th frame FRn+1a may not overlap. In this case, the fire determination unit 330 may determine that it is not a fire, indicating a false alarm.

In the case of fire, it may be classified into two types. The first type may be a fire that starts suddenly, has flames, and spreads quickly, while the second type may be a fire without flames that progresses slowly. The first type may be referred to as an “abrupt fire,” and the second type may be referred to as a “smoldering fire.”

Generally, the first type is accompanied by flames, making it easily detectable by the sensing unit SM, and the fire can be clearly detected using the first imaging unit CT1 and the second imaging unit CT2.

In the case of the second type of fire, the temperature may increase gradually. In this case, an accurate fire detection may be difficult with the sensing unit SM alone. According to an embodiment of the present disclosure, the gateway 300 may interpret the data received from the first imaging unit CT1 and the second imaging unit CT2 on a frame-by-frame basis to clearly detect the second type of fire. In other words, the first imaging unit CT1 and the second imaging unit CT2 may complement the sensing unit SM.

According to the present disclosure, the fire determination unit 330 may easily detect the second type of fire, which progresses slowly and generates little to no flame, using the first image data ID and the second image data IM2 measured in multiple frames. The fire determination unit 330 generates a fire event signal FE, and the origin positioning unit 340 may easily detect the fire through the fire event signal FE, and based on the detected fire, easily calculate the fire coordinates FC, which indicate the location where the fire has occurred. The server 400 can make a clear judgment regarding the fire and the location where the fire has occurred. Thus, the fire positioning system 10 with improved reliability can be provided.

According to the present disclosure, the gateway 300 may determine a false fire alarm before the server 400 makes the determination. Furthermore, the method of determining the false fire alarm at the gateway 300 may differ from the method used by the server 400. Therefore, the fire positioning system 10 with enhanced reliability can be provided.

Although the preferred embodiment of the present disclosure has been described with reference to the drawings, those skilled in the art or those with ordinary knowledge in the relevant field will understand that the present disclosure may be variously modified and changed within the scope of the claims without departing from the spirit and technical scope of the present disclosure. Therefore, the technical scope of the present disclosure should not be limited to the detailed description in the specification, but should be determined by the claims.

According to the above description, the gateway may include the origin positioning unit. The origin positioning unit can determine the location of the fire by using both a depth estimation algorithm and a back projection algorithm. The depth estimation algorithm can quickly estimate the distance between the fire and the first imaging unit by receiving the first image data in real-time. Since the back projection algorithm is based on simple calculations, it can track and recognize the fire in real-time, quickly calculating the fire coordinates. Through this, the origin positioning unit can calculate and output the location of the fire as the fire coordinates. The gateway can specify and output the location of the fire as the fire coordinates. The user or server can clearly recognize the location where the fire occurred and take appropriate follow-up actions. Therefore, the fire positioning system and the fire positioning method with improved reliability can be provided.

What is claimed is:

1. A fire positioning system comprising:
a sensing unit configured to measure temperature and smoke information to generate fire data;
a first imaging unit configured to capture an image to generate a first image;
a second imaging unit configured to capture an infrared image to generate a second image; and
a gateway configured to receive the fire data, the first image, and the second image,
wherein the gateway comprises:
a space coordinate definition unit configured to define a space where the sensing unit, the first imaging unit, and the second imaging unit are located in three-dimensional coordinates;
an image analysis unit configured to detect a fire based on the first image to generate first image data;
a fire determination unit configured to determine an occurrence of the fire based on the first image data and the second image to generate a fire event signal; and
an origin positioning unit configured to receive the first image data and the fire event signal,
wherein the origin positioning unit generates fire coordinates in accordance with the three-dimensional coordinates by using both a depth estimation algorithm and a back projection algorithm.

2. The fire positioning system of claim 1, wherein the depth estimation algorithm estimates a distance between the first imaging unit and a target by using a convolutional neural network.

3. The fire positioning system of claim 2, wherein the depth estimation algorithm estimates the distance by using the first image data for the first images.

4. The fire positioning system of claim 3, wherein the back projection algorithm generates the fire coordinates based on an optical center of the first imaging unit, the distance, and the three-dimensional coordinates.

5. The fire positioning system of claim 1, wherein the fire positioning system further comprises a server configured to communicate with the gateway,
wherein the gateway further comprises a communication unit and a power supply, and
wherein the communication unit transmits the fire event signal and the fire coordinates to the server.

6. The fire positioning system of claim 5, wherein the power supply is configured to supply power to the space coordinate definition unit, the fire determination unit, and the origin positioning unit.

7. The fire positioning system of claim 5, wherein the server calculates a fire index, which is an index of probability of a fire at a location corresponding to the fire coordinates based on the fire data, the first image, and the second image.

8. The fire positioning system of claim 1, wherein the fire determination unit determines a false fire alarm by comparing the first image data of an n-th frame with the first image data of an n+1-th frame.

9. The fire positioning system of claim 1, wherein the gateway receives the first image and the second image on a frame-by-frame basis, and
wherein the origin positioning unit calculates the fire coordinates for each frame.

10. The fire positioning system of claim 1, wherein the gateway further comprises a memory, and
wherein the memory stores location coordinates defined of the three-dimensional coordinates for each of the sensing unit, the first imaging unit, and the second imaging unit.

11. A fire positioning method comprising:
defining a space, in which a first imaging unit configured to capture an image to generate a first image and a second imaging unit configured to capture an infrared image to generate a second image are located, in three-dimensional coordinates;
detecting a fire based on the first image to generate first image data;
determining an occurrence of the fire based on the first image data and the second image to generate a fire event signal; and
receiving the first image data and the fire event signal, and generating fire coordinates based on the three-dimensional coordinates by using both a depth estimation algorithm and a back projection algorithm.

12. The fire positioning method of claim 11, wherein the depth estimation algorithm estimates a distance between the first imaging unit and a target by using a convolutional neural network.

13. The fire positioning method of claim 12, wherein the depth estimation algorithm estimates the distance by using the first image data for the first images.

14. The fire positioning method of claim 13, wherein the back projection algorithm generates the fire coordinates based on an optical center of the first imaging unit, the distance, and the three-dimensional coordinates.

15. The fire positioning method of claim 11, wherein generating the fire event signal further comprises determining a false fire alarm by comparing the first image data of an n-th frame with the first image data of an n+1-th frame.
